SEQUENCE LISTING (5 sequences)

SEQ ID NO: 1 - DNA, 35 bp, Artificial Sequence
  Description of Artificial Sequence Primer sequence
gcgaattcgccaccatgacttcgaaagtttatgat

SEQ ID NO: 2 - DNA, 29 bp, Artificial Sequence
  Description of Artificial Sequence Primer sequence
cactcgagttattgttcatttttgagaac

SEQ ID NO: 3 - DNA, 936 bp, Renilla reniformis
  CDS (1)..(936)
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagatcttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggatccaggattcttttccaatgctattgttgaaggcgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 4 - protein, 311 aa, Renilla reniformis
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 5 - DNA, 5446 bp, Artificial Sequence
  Description of Artificial Sequence pcDNA3 vector sequence
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2002/0192726 A1
(43) Pub. Date: Dec. 19, 2002

(54) Cell proliferation assay

(76) Inventor: Jay Leng, San Diego, CA (US)

Correspondence Address: Lisa A Haile PhD, Gray Cary Ware & Freidenrich LLP, 4365 Executive Drive, Suite 1100, San Diego, CA, 92121-2189, US

(21) Appl. No.: 09/559,874

(22) Filed: Apr. 25, 2000

(51) Int. Cl. G01N 33/574

(52) U.S. Cl. 435/7.23

(57) ABSTRACT

Disclosed are compositions and methods useful for measuring cell proliferation. The methods use a luciferase polypeptide to assess changes in cell number. Also disclosed are method to diagnose and monitor cell proliferative disorders including cancer. In addition, the methods disclosed are useful for identifying agents that modulate cell proliferation.

### FIELD OF THE INVENTION

The invention relates generally to cell proliferation and more particularly to cell proliferation assays using a luciferase.

### BACKGROUND

Control of cell proliferation is important in all multicellular organisms. Typically, cell number is determined, directly, by microscopic or electronic enumeration, or indirectly, by the use of chromogenic dyes, incorporation of radioactive precursors or measurement of metabolic activity of cellular enzymes. These methods are often insensitive, labor intensive, or hazardous. For example, measurement of cell proliferation generally involves the incorporation of a labeled nucleoside into genomic DNA. Examples include the tritiated thymidine (³H-dT) and bromodeoxyuridine (BrdU) methods. These techniques are of limited applicability, however, because of radiation induced DNA damage with the former and toxicities of nucleoside analogues with the latter.

One calorimetric assay is based on the cellular conversion of a tetrazolium salt into a blue formazan product that is detected using a ELISA plate reader (Mossmann T., J. Immunol, Meth. 65:55-63, 1983). Despite numerous attempts to reduce several technical problems related to this method (e.g., protein precipitation and incomplete solubilization of the formazan product), the assay is time consuming and requires the use of highly toxic materials (e.g., dimethyl formamide and thiazoyl blue).

There has been considerable interest recently in replacing radioactive labels used in analytical assays with other types of labels, such as luminescent labels. Firefly luciferase is one molecule that has been proposed for use as such labels. However, firefly luciferase suffers from a number of deficiencies that make this molecule less than optimal in biological assays. For example, ATP is required as an energy source in the firefly luciferase system, and the ubiquitous nature of ATP makes control of this variable difficult.

Indirect methods have also been used in specific cases. Recent interest in CD4⁺ T lymphocyte turnover in AIDS, for example, has been stimulated by indirect estimates of T cell proliferation based on their rate of accumulation in the circulation following initiation of effective anti-retroviral therapy (Ho et al., 1995, Nature 373:123-126; Wei et al., 1995, Nature 373:117-122). Unfortunately, such indirect techniques, which rely on changes in pool size, are not definitive. For example, the increase in the blood T cell pool size may reflect redistribution from other pools to blood rather than true proliferation. In the absence of direct measurements of cell proliferation, it may not be possible to distinguish between these and other alternatives.

Measurement of cell proliferation is of great diagnostic value in diseases such as cancer. Anti-cancer therapies aim to reduce tumor cell growth, which can be determined by whether tumor DNA is being synthesized or whether it is being broken down. Currently, the efficacy of therapy, whether chemotherapy, immunologic therapy or radiation therapy, is evaluated by indirect and often imprecise methods such as observation of apparent size of a tumor by x-ray. Efficacy of therapy and rational selection of combinations of therapies could be most directly determined on the basis of an individual tumor's biosynthetic and catabolic responsiveness to various interventions. One model used for bacterial infections in clinical medicine includes the culture of an organism and determination of its sensitivities to antibiotics followed by selection of an antibiotic to which the organism is sensitive can be used for cancer therapy as well. However, current management practices proceed without the ability to determine directly how well therapeutic agents are working.

A long-standing vision of oncologists is to be able to select chemotherapeutic agents similar to the way antibiotics are chosen, e.g. on the basis of measured sensitivity of tumor cells to a drug. The ability to measure cancer cell proliferation would place chemotherapy drug selection and research on an equal basis as antibiotic selection, with great potential for improved outcomes.

The Renilla, also known as sea pansies, belong to a class of coelenterates known as the anthozoans. In addition to Renilla, other representative bioluminescent genera of the class Anthozoa include Cavarnularia, Ptilosarcus, Stylatula, Acanthoptilum, and Parazoanthus. All of these organisms are bioluminescent and emit light as a result of the action of an enzyme (luciferase) on a substrate (luciferin) under appropriate biological conditions. Prior studies have demonstrated that all of the above-mentioned anthozoans contain similar luciferases and luciferins (See, for example, Cormier et al., J. Cell. Physiol. (1973) 81: 291-298). The luciferases and luciferins from each of these anthozoans crossreact with one another to produce the characteristic blue luminescence observed in Renilla extracts. Each of these luciferases has similar biochemical properties, and the biochemical requirements for bioluminescence are identical regardless of the anthozoan from which the luciferase was derived. The bioluminescence of Renilla luciferase lends itself to use in research and in vivo and in vitro assays.

### SUMMARY OF THE INVENTION

The present invention provides a method for determining the effect of an agent on cell proliferation by contacting a cell containing a Renilla luciferase polypeptide or a polynucleotide encoding a Renilla luciferase with an agent suspected of modulating cell proliferation under conditions that allow the agent and the cell to interact; and comparing the light emission data from the cell to the light emission data from the cell in the absence of the agent, wherein a difference in light emission data is indicative of an effect on cell proliferation. The cell may be a eukaryotic, a prokaryotic, or a plant cell, for example. In one aspect the cell is a cancer cell.

The invention also provides a method for determining cell proliferation of a cell or population of cells by obtaining light emission data from the cell(s) containing a Renilla luciferase over a period of time wherein a change in light emission data is indicative of proliferation or a change in cell number.

The invention further provides a method for determining the effect of an agent on cell proliferation. The method includes transfecting a cell with a vector containing a polynucleotide sequence encoding a Renilla luciferase; contacting the transfected cell with an agent suspected of modulating cell proliferation under conditions that allow the agent and the cell to interact; and comparing the light emission data from the cell to the light emission data from the cell in the absence of the agent, wherein a difference in light emission data is indicative of an effect on cell proliferation. The cell can be any cell such as a prokaryotic cell or a eukaryotic cell, e.g., a mammalian cell or a human cell. In one aspect the cell is a cancer cell.

The invention also provides a eukaryotic host cell containing an expression vector encoding Renilla luciferase. For example, the host cell can be any eukaryotic or mammalian cell such as a human cell. In one aspect, the cell is a HeLa cell. In a further embodiment, the HeLa cell has ATCC accession number X. In yet another aspect, the cell is a plant cell.

The invention also provides a method of diagnosing a cell proliferative disorder in a subject or in a population of cells by transfecting a cell obtained from the subject with a polynucleotide encoding a Renilla luciferase; obtaining light emission data from the cell over a period of time; and comparing the light emission data from the cell to light emission data from a cell which does not have a cell proliferative disorder, wherein a difference in light emission is indicative of a cell proliferative disorder.

Also provided is a method of screening eukaryotic cells to determine their susceptibility to treatment with an agent. The method includes contacting cells containing a Renilla luciferase with an agent and measuring light emissions from the cells in the presence and absence of the agent, wherein a difference in light emissions is indicative of an agent which affects cell proliferation.

In another embodiment, the invention provides a kit comprising a container containing a eukaryotic or a plant host cell containing a Renilla luciferase and instructions for use of the cell for measuring cell proliferation.

In another embodiment, the invention provides genetic material encoding Renilla luciferase. The genetic material can be used to produce the enzyme for use as luminescent tags in bioluminescence assays and for other purposes for which such labels are desirable. Accordingly, in one embodiment, the invention provides a vector containing a polynucleotide sequence encoding a Renilla luciferase for expression in a eukaryotic organism or a plant cell.

### BRIEF DESCRIPTION OF THE FIGURES

FIG. 1 (A-B) shows a sequence (SEQ ID Nos:3-5) and (C) a vector map of a renilla luciferase.

FIG. 2 shows a cell proliferation assay comparing the sensitivity of an MTT assay to the sensitivity of a luciferase assay of the invention.

FIG. 3 shows a cell proliferation assay of a transformed HeLa cell of the invention compared to an MTT assay of HeLa cells.

### DETAILED DESCRIPTION OF THE INVENTION

As used herein and in the appended claims, the singular forms “a,” “and,” and “the” include plural referents unless the context clearly dictates otherwise. Thus, for example, reference to “a sample” includes a plurality of samples and reference to “the agent” generally includes reference to one or more agents and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference in full for the purpose of describing and disclosing the databases, proteins, and methodologies, which are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The headings and subheadings used herein are for the convenience of the reader and are not intended to limit the invention.

Renilla Luciferase

As used herein, “Renilla luciferase” means a luciferase enzyme isolated from a member of the genus Renilla or an equivalent molecule obtained from any other source or obtained synthetically.

The photoprotein aequorin (which consists of apoaequorin bound to a coelenterate luciferin molecule) and Renilla luciferase both utilize the same coelenterate luciferin, and the chemistry of light emission in both cases has been shown to be the same. However, aequorin luminescence is triggered by calcium and represents a single turnover event. In contrast, Renilla luciferase is not triggered by calcium and requires dissolved oxygen in order to produce light in the presence of coelenterate luciferin. Renilla luciferase also acts as a true enzyme, catalyzing a long-lasting luminescence in the presence of saturating levels of luciferin.

Sub-attomole levels of aequorin can be detected with photometers even though its luminescence represents a single turnover event. Renilla luciferase, due to its enzymatic ability, is detectable at levels one to two orders of magnitude lower than aequorin. Furthermore, Renilla luciferase is known to be relatively stable to heat, an important consideration for assays that often involve incubation at physiological temperatures. Accordingly, Renilla luciferase is a desirable tool for biological and other assays.

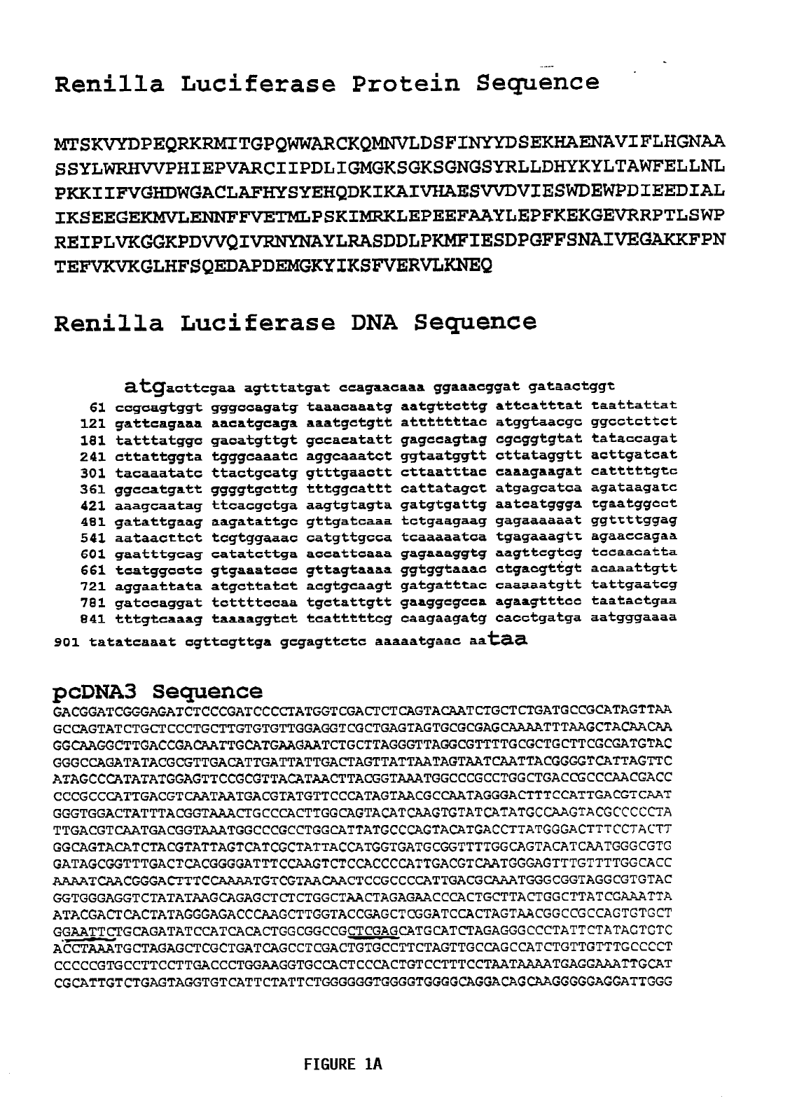

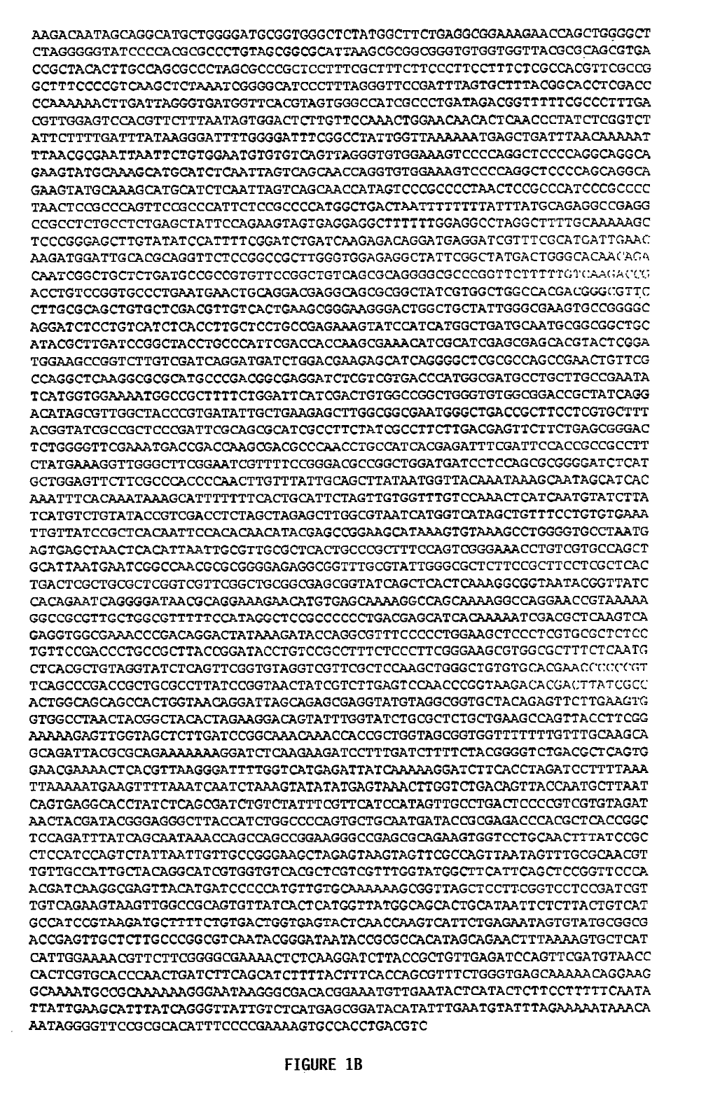

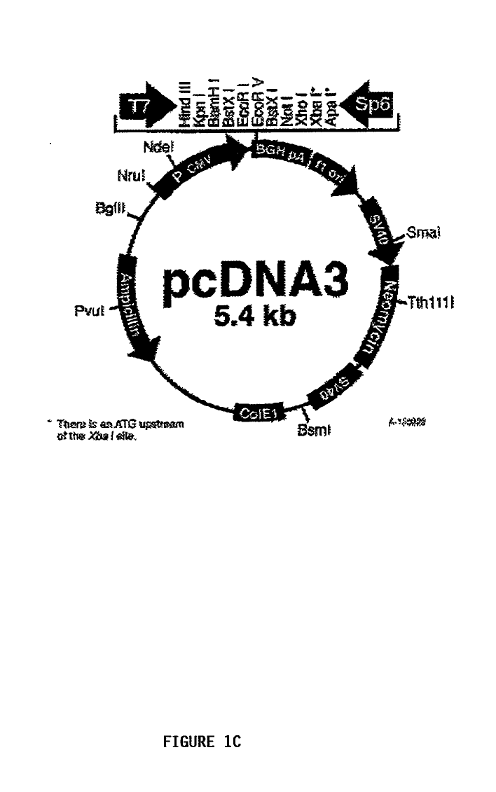

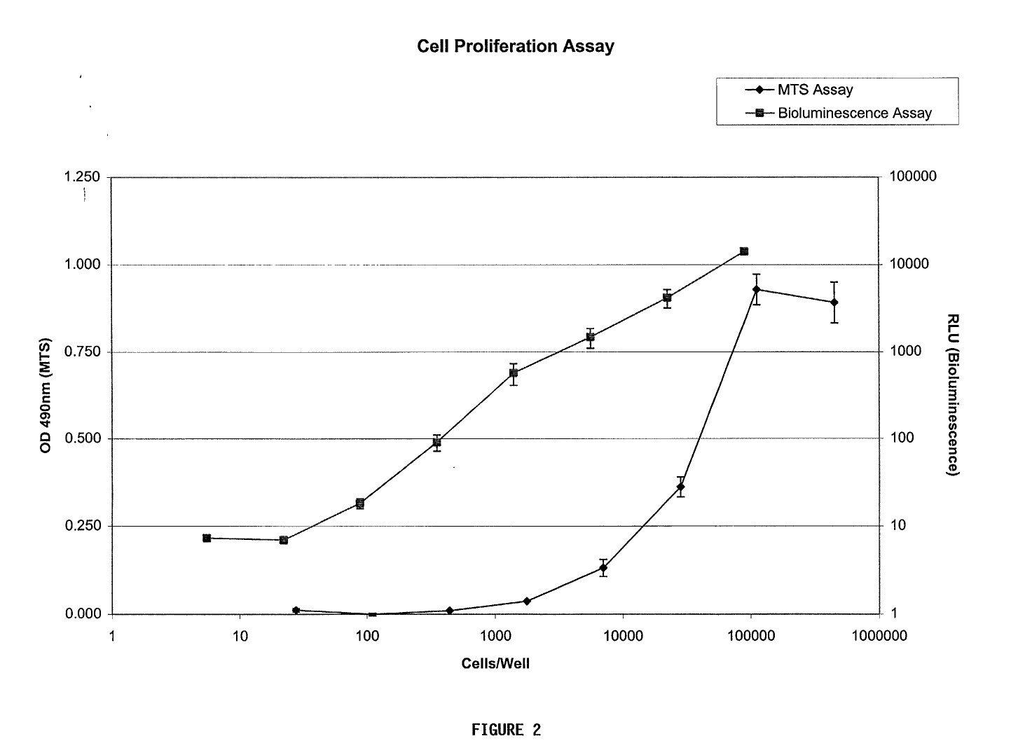

On the other hand, Renilla live on the ocean bottom, about 30 to 100 feet deep, and must be collected by dredging. From 1 kg of Renilla (about 1000 individual animals), approximately 1 mg of pure Renilla luciferase can be obtained following a tedious procedure which requires purifying the protein about 12,000 fold. The purification procedure is described in Matthews et al. (Biochemistry, 16: 85-91, 1977). Renilla luciferase has been cloned, sequenced and expressed as described in U.S. Pat. Nos. 5,292,658 and 5,418,155, the disclosures of which are incorporated herein by reference. FIGS. 1 and 2 of U.S. Pat. No. 5,292,658 disclose the polynucleotide sequence and corresponding amino acid sequence of a Renilla luciferase.

Since a polynucleotide sequence of the Renilla luciferase has been identified, it is possible to produce a polynucleotide sequence entirely by synthetic chemistry, after which the polynucleotide can be inserted into any of the many available DNA vectors using known techniques of recombinant DNA technology. Thus, the present invention can be carried out using reagents, plasmids, and microorganisms which are freely available and in the public domain at the time of filing of this patent application without requiring a deposit of genetic material.

Polynucleotide or nucleic acid sequence refers to a polymeric form of nucleotides. In some instances a polynucleotide refers to a sequence that is not immediately contiguous with either of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. In addition, the polynucleotide sequence involved in producing a polypeptide chain can include regions preceding and following the coding region (leader and trailer) as well as intervening sequences (introns) between individual coding segments (exons) depending upon the source of the polynucleotide sequence. In addition, polynucleotides greater than 100 bases long can be readily synthesized on an Applied Biosystems Model 380A DNA Synthesizer, for example.

The term polynucleotide(s) generally refers to any polyribonucleotide or polydeoxyribonucleotide, which may be unmodified RNA or DNA or modified RNA or DNA. Thus, for instance, polynucleotides as used herein refers to, among others, single-and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

In addition, the polynucleotides or nucleic acid sequences may contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

Nucleic acid sequences can be created which encode a fusion protein and can be operatively linked to expression control sequences. “Operatively linked” refers to a juxtaposition wherein the components so described are in a relationship permitting them to function in their intended manner. For example, a coding sequence is “operably linked” to another coding sequence when RNA polymerase will transcribe the two coding sequences into a single mRNA, which is then translated into a single polypeptide having amino acids derived from both coding sequences. The coding sequences need not be contiguous to one another so long as the expressed sequences ultimately process to produce the desired protein. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

By “promoter” is meant minimal sequence sufficient to direct transcription. Also included in the invention are those promoter elements which are sufficient to render promoter-dependent gene expression controllable for cell-type specific, tissue-specific, or inducible by external signals or agents; such elements may be located in the 5′ or 3′ regions of the of the polynucleotide sequence. Both constitutive and inducible promoters, are included in the invention (see e.g., Bitter et al., Methods in Enzymology 153:516-544, 1987). For example, when cloning in bacterial systems, inducible promoters such as pL of bacteriophage, plac, ptrp, ptac (ptrp-lac hybrid promoter) and the like may be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used to provide for transcription of the nucleic acid sequences of the invention. When cloning in plant systems plant promoters can be used with the construct comprising a plant promoter operably associated with a sequence encoding a luciferase. The plant promoter may be constitutive or inducible. Useful constitutive promoters include, but are not limited to, the CaMV 35S promoter, the T-DNA mannopine synthetase promoter, and their various derivatives. Useful inducible promoters include but are not limited to the promoters of ribulose bisphosphate carboxylase (RUBISCO) genes, chlorophyll binding protein (CAB) genes, heat shock genes, the defense responsive gene (e.g., phenylalanine ammonia lyase genes), wound induced genes (e.g., hydroxyproline rich cell wall protein genes), chemically-inducible genes (e.g., nitrate reductase genes, gluconase genes, chitinase genes, PR-1 genes), dark-inducible genes (e.g., asparagine synthetase gene (Coruzzi and Tsai, U.S. Pat. No. 5,256,558) to name just a few.

A nucleic acid sequence of the invention including, for example, a polynucleotide encoding a fusion protein, may be inserted into a recombinant expression vector. A recombinant expression vector generally refers to a plasmid, virus or other vehicle known in the art that has been manipulated by insertion or incorporation of a nucleic acid sequences. For example, a recombinant expression vector of the invention includes a polynucleotide sequence encoding a Renilla luciferase polypeptide of fragment thereof. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al., Gene 56:125, 1987), the pMSXND expression vector for expression in mammalian cells (Lee and Nathans, J. Biol. Chem. 263:3521, 1988), baculovirus-derived vectors for expression in insect cells, cauliflower mosaic virus, CaMV; tobacco mosaic virus, TMV. The nucleic acid sequences of the invention can also include a localization sequence to direct the indicator to particular cellular sites by fusion to appropriate organellar targeting signals or localized host proteins. For example, a polynucleotide encoding a localization sequence, or signal sequence, can be used as a repressor and thus can be ligated or fused at the 5′ terminus of a polynucleotide encoding a polypeptide of the invention such that the localization or signal peptide is located at the amino terminal end of a resulting polynucleotide/polypeptide (see for example, Liu et al., Gene, 203(2):141-8, 1997). The construction of expression vectors and the expression of genes in transfected cells involves the use of molecular cloning techniques also well known in the art. (See, for example, Sambrook et al., Molecular Cloning—A Laboratory Manual, Cold Spring Harbor Laboratory, Cold Spring Harbor, N.Y., 1989, and Current Protocols in Molecular Biology, M. Ausubel et al., eds., (Current Protocols, a joint venture between Greene Publishing Associates, Inc. and John Wiley & Sons, Inc., most recent Supplement)). These methods include in vitro recombinant DNA techniques, synthetic techniques and in vivo recombination/genetic recombination. (See also, Maniatis, et al., Molecular Cloning A Laboratory Manual, Cold Spring Harbor Laboratory, N.Y., 1989).

In yeast, a number of vectors containing constitutive or inducible promoters may be used. For a review see, Current Protocols in Molecular Biology, Vol. 2, Ed. Ausubel, et al., Greene Publish. Assoc. & Wiley Interscience, Ch. 13, 1988; Grant, et al., “Expression and Secretion Vectors for Yeast,” in Methods in Enzymology, Eds. Wu & Grossman, 1987, Acad.

Press, N.Y., Vol. 153, pp.516-544, 1987; Glover, DNA Cloning, Vol. II, IRL Press, Wash., D.C., Ch. 3, 1986; and Bitter, “Heterologous Gene Expression in Yeast,” Methods in Enzymology, Eds. Berger & Kimmel, Acad. Press, N.Y., Vol. 152, pp. 673-684, 1987; and The Molecular Biology of the Yeast Saccharomyces, Eds. Strathern et al., Cold Spring Harbor Press, Vols. I and U, 1982. A constitutive yeast promoter such as ADH or LEU2 or an inducible promoter such as GAL may be used (“Cloning in Yeast,” Ch. 3, R. Rothstein In: DNA Cloning Vol. 1 1, A Practical Approach, Ed. DM Glover, IRL Press, Wash., D.C., 1986). Alternatively, vectors may be used which promote integration of foreign DNA sequences into the yeast chromosome.

An alternative expression system which could be used to express a Renilla luciferase polypeptide of the invention is an insect system. In one such system, Autographa californica nuclear polyhedrosis virus (AcNPV) is used as a vector to express foreign or mutated polynucleotide sequences. The virus grows in Spodoptera frugiperda cells. The sequence encoding a protein of the invention may be cloned into non-essential regions (for example, the polyhedrin gene) of the virus and placed under control of an AcNPV promoter (for example the polyhedrin promoter). Successful insertion of the sequences coding for a protein of the invention will result in inactivation of the polyhedrin gene and production of non-occluded recombinant virus (i.e., virus lacking the proteinaceous coat coded for by the polyhedrin gene). These recombinant viruses are then used to infect S. frugiperda cells in which the inserted gene is expressed, see Smith, et al., J. Viol. 46:584, 1983; Smith, U.S. Pat. No. 4,215,051.

The vectors of the invention can be used to transform a host cell. By transform or transformation is meant a permanent or transient genetic change induced in a cell following incorporation of new DNA (i.e., DNA exogenous to the cell). Where the cell is a mammalian cell, a permanent genetic change is generally achieved by introduction of the DNA into the genome of the cell.

A transformed cell or host cell generally refers to a cell (e.g., prokaryotic, eukaryotic or plant cells) into which (or into an ancestor of which) has been introduced, by means of recombinant DNA techniques, a DNA molecule encoding a Renilla luciferase polypeptide or fragment thereof. The term “plant cell” as used herein refers to protoplasts, gamete producing cells, and cells which regenerate into whole plants. Plant cells include cells in plants as well as protoplasts in culture. Accordingly, a seed comprising multiple plant cells capable of regenerating into a whole plant, is included in the definition of “plant cell”.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method by procedures well known in the art. Alternatively, MgCl₂ or RbCl can be used. Transformation can also be performed after forming a protoplast of the host cell or by electroporation.

When the host is a eukaryote, methods of transfection or transformation with DNA include calcium phosphate co-precipitates, conventional mechanical procedures such as microinjection, electroporation, insertion of a plasmid encased in liposomes, or virus vectors, as well as others known in the art, may be used. Eukaryotic cells can also be cotransfected with DNA sequences encoding a Renilla luciferase polypeptide and a second foreign DNA molecule encoding a selectable marker, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Typically, a eukaryotic host will be utilized as the host cell. The eukaryotic cell may be a yeast cell (e.g., Saccharomyces cerevisiae), an insect cell (e.g., Drosophila sp.) or may be a mammalian cell, including a human cell.

Eukaryotic systems, and mammalian expression systems, allow for post-translational modifications of expressed mammalian proteins to occur. Eukaryotic cells which possess the cellular machinery for processing of the primary transcript, glycosylation, phosphorylation, and, advantageously secretion of the gene product should be used. Such host cell lines may include, but are not limited to, CHO, VERO, BHK, HeLa, COS, MDCK, Jurkat, HEK-293, and W138.

Mammalian cell systems which utilize recombinant viruses or viral elements to direct expression may be engineered. For example, when using adenovirus expression vectors, a polynucleotide encoding a Renilla luciferase may be ligated to an adenovirus transcription/ translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a Renilla luciferase polypeptide or fragment thereof in infected hosts (e.g., see Logan & Shenk, Proc. Natl. Acad. Sci. USA, 81:3655-3659, 1984). Alternatively, the vaccinia virus 7.5K promoter may be used. (e.g., see, Mackett, et al., Proc. Natl. Acad. Sci. USA, 79:7415-7419, 1982; Mackett, et al., J. Virol. 49:857-864, 1984; Panicali, et al., Proc. Natl. Acad. Sci. USA 79:4927-4931, 1982). Of particular interest are vectors based on bovine papilloma virus which have the ability to replicate as extrachromosomal elements (Sarver, et al., Mol. Cell. Biol. 1:486, 1981). Shortly after entry of this DNA into mouse cells, the plasmid replicates to about 100 to 200 copies per cell. Transcription of the inserted cDNA does not require integration of the plasmid into the host's chromosome, thereby yielding a high level of expression. These vectors can be used for stable expression by including a selectable marker in the plasmid, such as the neo gene. Alternatively, the retroviral genome can be modified for use as a vector capable of introducing and directing the expression of a Renilla luciferase gene in host cells (Cone & Mulligan, Proc. Natl. Acad. Sci. USA, 81:6349-6353, 1984). High level expression may also be achieved using inducible promoters, including, but not limited to, the metallothionine IIA promoter and heat shock promoters.

For long-term, high-yield production of recombinant proteins, stable expression is preferred. Rather than using expression vectors which contain viral origins of replication, host cells can be transformed with the cDNA encoding a Renilla luciferase polypeptide controlled by appropriate expression control elements (e.g., promoter, enhancer, sequences, transcription terminators, polyadenylation sites, etc.), and a selectable marker. The selectable marker in the recombinant vector confers resistance to the selection and allows cells to stably integrate the plasmid into their chromosomes and grow to form foci which in turn can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase (Wigler, et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy, et al., Cell, 22:817, 1980) genes can be employed in tk-, hgprt- or aprt- cells respectively. Also, anti-metabolite resistance can be used as the basis of selection for dhfr, which confers resistance to methotrexate (Wigler, et al., Proc. Natl. Acad. Sci. USA, 77:3567, 1980; O'Hare, et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin, et al., J. Mol. Biol. 150:1, 1981); and hygro, which confers resistance to hygromycin (Santerre, et al., Gene 30:147, 1984) genes. Recently, additional selectable genes have been described, namely trpB, which allows cells to utilize indole in place of tryptophan; hisd, which allows cells to utilize histinol in place of histidine (Hartman & Mulligan, Proc. Natl. Acad. Sci. USA 85:8047, 198 8); and ODC (ornithine decarboxylase) which confers resistance to the ornithine decarboxylase inhibitor, 2-(difluoromethyl)-DL-omithine, DFMO (McConlogue L., In: Current Communications in Molecular Biology, Cold Spring Harbor Laboratory, ed., 1987).

The invention provides a stably transfected mammalian cell containing a luciferase gene useful in measuring cell proliferation and the affect of an agent on cell proliferation. A HeLa cell was stably transfected with a Renilla luciferase gene as described in the examples below. This cell was expanded for cryopreservation. A sample of this cell line has been deposited in the American Type Culture Collection, Rockville, Md., U.S.A. under the provisions of the Budapest Treaty and assigned accession number ATCC XXXX.

The term “primer” as used herein refers to an oligonucleotide, whether natural or synthetic, which is capable of acting as a point of initiation of synthesis when placed under conditions in which primer extension is initiated or possible. Synthesis of a primer extension product which is complementary to a nucleic acid strand is initiated in the presence of nucleoside triphosphates and a polymerase in an appropriate buffer at a suitable temperature. For instance, if a nucleic acid sequence is inferred from a protein sequence, a primer generated to synthesize nucleic acid sequence encoding the protein sequence is actually a collection of primer oligonucleotides containing sequences representing all possible codon variations based on the degeneracy of the genetic code. One or more of the primers in this collection will be homologous with the end of the target sequence. Likewise, if a “conserved” region shows significant levels of polymorphism in a population, mixtures of primers can be prepared that will amplify adjacent sequences. For example, primers can be synthesized based upon the nucleotide or amino acid sequence of Renilla luciferase such as those set forth in SEQ ID NO: 1 or 2, respectively. Where the amino acid sequence is used, the primer sequence can be designed based upon the degeneracy of the genetic code.

A polypeptide or protein refers to a polymer in which the monomers are amino acid residues which are joined together through amide bonds. When the amino acids are alpha-amino acids, either the L-optical isomer or the D-optical isomer can be used, the L-isomers being typical. A Renilla luciferase polypeptide is intended to encompass any amino acid sequence and include modified sequences such as glycoproteins, which provides a polypeptide having luciferase activity. Accordingly, the polypeptides of the invention are intended to cover naturally occurring proteins, as well as those which are recombinantly or synthetically synthesized. In addition, a Renilla luciferase polypeptide can occur in at least two different conformations wherein both conformations have the same or substantially the same amino acid sequence but have different three dimensional structures so long as the have a biological activity related to Renilla luciferase. Polypeptide or protein fragments of Renilla luciferase are also encompassed by the invention so long as they retain some activity of a full-length luciferase (e.g., at least about 50%, 75%, 100% or more of such activity). Fragments can have the same or substantially the same amino acid sequence as the naturally occurring protein. A polypeptide or peptide having substantially the same sequence means that an amino acid sequence is largely, but not entirely, the same, but retains a functional activity of the sequence to which it is related. In general polypeptides of the present invention include peptides, or full length protein, that contains substitutions, deletions, or insertions into the protein backbone, that would still have an approximately 70%-90% homology to the original protein over the corresponding portion. A yet greater degree of departure from homology is allowed if like-amino acids, i.e. conservative amino acid substitutions, do not count as a change in the sequence.

Homology can be measured using standard sequence analysis software (e.g., Sequence Analysis Software Package of the Genetics Computer Group, University of Wisconsin Biotechnology Center, 1710 University Avenue, Madison, Wis. 53705; also see Ausubel, et al., supra). Such procedures and algorithms include, for example, a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF.

A polypeptide is substantially related but for a conservative variation, such polypeptides being encompassed by the present invention. A conservative variation denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

Modifications and substitutions are not limited to replacement of amino acids. For a variety of purposes, such as increased stability, solubility, or configuration concerns, one skilled in the art will recognize the need to introduce, (by deletion, replacement, or addition) other modifications. Examples of such other modifications include incorporation of rare amino acids, dextra-amino acids, glycosylation sites, cytosine for specific disulfide bridge formation. The modified peptides can be chemically synthesized, or the isolated gene can be site-directed mutagenized, or a synthetic gene can be synthesized and expressed in bacteria, yeast, baculovirus, tissue culture and so on. Whether a change results in a functioning peptide can readily be determined by direct analysis for function in a assay that relies on ability of the modified enzyme (or fragment) to carry out the normal function of the natural luciferase enzyme (or fragment). For example, modified peptides can be tested for ability to catalyze the emission of light from coelenterate luciferin by the same techniques described below for the recombinant Renilla luciferase molecule. Alternatively, the modified sequences can be screened for functional activity by attaching a suitable substrate, e.g., a coelenterate luciferin molecule, to an affinity column and capturing modified peptides that are retained by the bound substrate.

Solid-phase chemical peptide synthesis methods can also be used to synthesize the polypeptide or fragments of the invention. Such method have been known in the art since the early 1960's (Merrifield, R. B., J. Am. Chem. Soc., 85, 2149-2154 (1963) (See also Stewart, J. M. and Young, J. D., Solid Phase Peptide Synthesis, 2 ed., Pierce Chemical Co., Rockford, Ill., pp. 11-12)) and have recently been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81, 3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available. For example, assembly of a polypeptide or fragment can be carried out on a solid support using an Applied Biosystems, Inc. Model 431A automated peptide synthesizer. Such equipment provides ready access to the peptides of the invention, either by direct synthesis or by synthesis of a series of fragments that can be coupled using other known techniques.

Functional fragments of a Renilla luciferase, based on these sequences and fragments and full length sequences representing minor variations thereof, will have at least some of the biological activities of luciferase and will therefore be useful in appropriate circumstances. For example, functional fragments of the luciferase enzyme sequence can be prepared and screened for use as luciferin binding site models. Peptide synthesizers (as described above) can be used to prepare peptide fragments (e.g., less than 100 amino acids) or techniques of genetic engineering can be used to prepare the peptide fragments. The fragments can then be screened for functional activity by attaching a suitable substrate, e.g., a coelenterate luciferin molecule, to an affinity column and capturing peptide fragments that are retained by the bound substrate. Polypeptides or fragments that retain at least about 50% activity of luciferase are encompassed by the invention.

Pathologies Associated with Cell Proliferative Disorders

A number of diseases or disorders are known to be characterized by altered cellular proliferation rates and thus can be monitored, diagnosed or used in the development of therapies for an afflicted subject. As used herein, “subject” means any mammal, but is preferably a human. For example, cancer and malignant tumors of any type, including breast cancer, lung cancer, colon cancer, skin cancer, lymphomas, and leukemias; pre-cancerous conditions such as adenomas, polyps, prostatic hypertrophy, and ulcerative colitis can be diagnosed using the methods of the invention. Immune disorders associated CD4+ and CD8+T lymphocytes in AIDS; T and B lymphocytes in vaccine-unresponsiveness; T cells in autoimmune disorders; B cells in hypogammaglobulinemias; primary immunodeficiencies (thymocytes); stress-related immune deficiencies (lymphocytes) and the like can also be diagnosed by the method of the invention. Other disorders include, for example, hematologic conditions (e.g., white blood cell deficiencies (granulocytopenia), anemias of any type, myeloproliferative disorders (polycythemia vera), tissue white cell infiltrative disorders (pulmonary interstitial eosinophilia, lymphocytic thyroiditis, etc.), lymphoproliferative disorders, monoclonal gammopathies; and the like); organ failure (e.g., alcoholic and viral hepatitis (liver cells), diabetic nephropathy (glomerular or mesangeal cells), myotrophic conditions (myocytes), premature gonadal failure (oocytes, stromal cells of ovary, spermatocytes, Leydig cells, etc.), and the like); conditions of bone and muscle (e.g., response to exercise training or physical therapy (myocytes or mitochondria in myocytes), osteoporosis (osteoclast, osteoblasts, parathyroid cells) myositis, and the like); endocrine conditions (e.g., diabetes (β islet-cells), hypothyroidism and hyperthyroidism (thyroid cells), hyperparathyroidism (parathyroid cells), polycystic ovaries (stromal cells of ovary), and the like); infectious diseases (e.g., tuberculosis (monocytes/macrophages), bacterial infections (granulocytes), abscesses and other localized tissue infections (granulocytes), viral infections (lymphocytes), diabetes foot disease and gangrene (white cells), and the like); vascular disorders such as, for example, atherogenesis (smooth muscle proliferation in arterial wall), cardiomyopathies (cardiac myocyte proliferation), and the like; and occupational diseases and exposures including susceptibility to coal dust for black lung (fibroblast proliferative response), susceptibility to skin disorders related to sun or chemical exposures (skin cells), and the like.

Methods of Measuring Cell Proliferation

The methods provided herein, are well-suited for measuring any change in the number of cells in culture. The invention provides methods of measuring cell proliferation of a cell or population of cells by obtaining light emission data from a cell containing a Renilla luciferase over a period of time, wherein a change in light emission data is indicative of proliferation or a change in cell number. Such methods can provide information regarding the rate at which a cell is proliferating, the rate at which the population of cells in culture changes, as well as information regarding the total number of cells in culture.

The cell may be any cell including prokaryotic and eukaryotic cells (e.g., invertebrate, vertebrate, mammalian and human cells, or plant cells). The methods herein are particularly adaptable to determination of the change in cell number of a cell or a culture of cells such as in microwell tissue culture plates. These plates typically have 96 wells per plate, but higher density formats are available, including the 96 well half area format, and 384 well plates, and can accommodate as little as a fraction of a milliliter of cells or cell lysate per well, for example, 0.2 ml or less, containing as few as 50 cells. Use of such plates are advantageously adaptable to automation. Cells are introduced into the microtiter tissue culture plates or other suitable vessels. The cells can be adherent cells or grow in suspension. The cells can be transfected with a Renilla luciferase (as described above) either before primary culture or during subculture. Such transfected cells can be transiently transfected or stably transfected depending upon the culture conditions and assay conditions, such techniques for stable and transient transfections are known in the art (as described above). The cells may be obtained from any sample, including soil, water, or a biological sample (e.g., blood, urine, sputum, spinal fluid, and tissue).

The cells containing a Renilla luciferase are cultured under conditions that allow expression of the Renilla luciferase. The luciferase activity can then be measured in vivo or in vitro (see, for example, Lorenzo et al., J Biolumin Chemilumin, 11(1):31-7, 1996, which is incorporated by reference herein) by providing the cell culture with the substrate coelentrazine. Typically the coelentrazine will be in an amount of about 0.05 μM to about 5 μM, depending, for example, upon the assay conditions (e.g., whole cell, lysate, purified protein). Alternatively, the cells can be lysed prior to addition of the substrate. In such instances the cells can be lysed by adding appropriate buffer or by mechanical disruption or other methods known to those of skill in this art. The vessels, particularly the microtiter plates, can be placed in commercially available instruments for measuring light, such as a plate reader, which can be interfaced with a computer for data analysis. Depending upon the assay type, one skilled in the art can develop various methods to determine a change in cell number. For example, where cell death is measured, the cells can be washed between measurements to determine the number of cells or luciferase activity present before and after the wash. For example, a decrease in the number of cells over a period of time is indicative of cell death.

The determination of the number of cells can be made at any desired time during a cell culture period in which the growth, survival, or death of the cells are to be measured. These cells can be grown in conventional tissue culture flasks such as, for example, T-flasks, roller bottles, flat bed chambers, hollow fiber reactors, agitated suspension culture vessels and similar cell culture devices under various suspension or anchorage-dependent cell culture conditions. In one embodiment, microtiter plates designed for use with high throughput automated instrumentation can be used. The time and frequency of the cell counts will depend upon the nature of the specific cells being cultured, their normal growth period, the cell products sought after and other such factors. For example, measurements of luciferase activity can be measured continuously or at different time points (e.g., 0 sec., 1 minute, 2 minutes, 5 minutes, 10 minutes, 20 minutes and so on). By following a cell count, one can readily determine the growth phase or stage in which the cells exist at any given time.

Cell number can by determined by comparing the light produced by cells with a control population of cells or a control curve developed using identical conditions, but a known or predetermined number of cells. A curve can be developed, for example, by determining the number of cells per well or other unit of cells in at least two different levels and plotting against light or luciferase activity to form a straight line relationship. The number of cells for any cell culture of the given cells can then be estimated by carrying out the method and comparing light emission or luciferase activity to a control curve. In automated methods, such data can be included in the programming of the instrument and cell number determined automatically. Such methods can assist in the diagnoses of a cell proliferative disorder. For example, cells isolated from a subject suspected of having a cell proliferative disorder (e.g., a cancer or neoplasm) can be compared to control cells in order to determine if the proliferative rates of the cancer cells are different than normal or non-cancerous cells. The control or non-cancerous cells may be from the same subject, for example, from a different tissue site.

The methods of the invention are also useful in monitoring the treatment of a subject diagnosed with a cell proliferative disorder. In this embodiment, the cells of a subject are monitored following or concurrent with the beginning of a course of therapeutic treatment. Such therapeutic treatments include, for example the administration of chemotherapeutic agents where cancer has been diagnosed or bone marrow transplantation following bone marrow ablation or treatment with a chemotherapeutic agent. For example, a subject diagnosed with a cancer can have biopsies taken at various intervals of time to determine whether the cells present in the biopsy contain cancer cells by measuring cell proliferation as described above. Similarly, cells can be taken from the bone marrow of a subject to determine whether bone marrow replacement therapy is working by measuring whether the bone marrow cells in the biopsy are viable and proliferating, for example.

The Examples described below demonstrate that the methods herein can be adapted for use with a wide variety of cell types, and, can, thus be used for any purpose in which cell proliferation or cell number is measured. Because of the high sensitivity of the method, it is particularly adaptable for use in assays for screening for compounds that modulate cell proliferation (e.g., drug screening assays). Inhibitors of proliferation will be useful for treating pathologies that derive from cell proliferation, such as tumors, diabetic retinopathies, arthritis and other such disorders described herein. Identification of stimulators of cell proliferation is also of interest as a means to identify transformation factors.

Methods of Identifying Agents that Modulate Cell Proliferation

In another embodiment, the invention provides a method for identifying an agent which modulates cell proliferation or activity including incubating components comprising the agent and a cell containing a Renilla luciferase polypeptide, or a recombinant cell expressing a Renilla luciferase polypeptide, under conditions sufficient to allow the components to interact and determining the effect of the agent on cell proliferation or activity. The term “effect”, as used herein, encompasses any means by which cell proliferation or activity can be modulated such as inhibition or stimulation of cell proliferation. “Agents” can include, for example, polypeptides, peptidomimetics, chemical compounds and biologic agents as described below.

Incubating includes conditions which allow contact between the test agent and cell containing a Renilla luciferase, a cell expressing a Renilla luciferase or a sample containing a Renilla luciferase. Contacting includes in solution and in solid phase. The test agents may optionally be a combinatorial library for screening a plurality of agents. Agents identified in the method of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 3:1008-1012, 1985), oligonucleotide ligation assays (OLAs) (Landegren, et al., Science, 241:1077, 1988), and the like. Molecular techniques for DNA analysis have been reviewed (Landegren, et al., Science, 242:229-237, 1988).

Thus, the method of the invention includes combinatorial chemistry methods for identifying agents that modulate cell proliferation or affect cell proliferation or activity. Areas of investigation are the development of therapeutic treatments for cancer or other disorders associated with abnormal cellular proliferation. The screening identifies agents that modulate cell proliferation by either stimulating cell proliferation or inhibiting cell proliferation. Of particular interest are screening assays for agents that have a low toxicity for human cells. For example, chemotherapeutic agents that inhibit cells having cell proliferative disorders while maintaining proliferation of normal cells. In addition, screening antibiotic agents to determine effective antibiotics for a particular microorganism is applicable to the methods of the invention.

The term “agent” as used herein describes any molecule, e.g. protein or pharmaceutical, with the capability of altering cell proliferation (e.g., stimulating or inhibiting cell proliferation). Generally, a plurality of assay mixtures are run in parallel with different agent concentrations to obtain a differential response to the various concentrations. Typically, one of these concentrations serves as a negative control, e.g. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification and amidification to produce structural analogs.

A variety of other reagents may be included in the screening assay. These include reagents like salts, neutral proteins, e.g. albumin, detergents, etc that are used to facilitate optimal agent cell interaction, stability of the agent being tested and/or reduce non-specific or background interactions. Reagents that improve the efficiency of the assay, such as protease inhibitors, nuclease inhibitors and anti-microbial agents may be used. Incubations are performed at any suitable temperature, typically between 4 and 50° C. Incubation periods are selected for optimum activity, but may also be optimized to facilitate rapid high-throughput screening. Between 0.1 and 48 hours, typically 1-24 hours, but more typically 1-12 hours will be sufficient time to measure proliferation.

The invention now being generally described, the same will be better understood by reference to the following examples which are provided for purposes of illustration only and are not to be considered limiting of the invention unless so specified.

### EXAMPLES

Cloning of Renilla Luciferase

A SeaLite plasmid pCR3.1 (SeaLite Sciences, Inc., Norcross, Ga.) was amplified by PCR with oligonucleotide primers containing Kozak sequences at it N-terminal primer:

LucP4-EcoRI: GCGAATTCGCCACCATGACTTCGAAAGTTTATGAT (SEQ ID NO: 1); and

LucP2-XhoI: CACTCGAGTTATTGTTCATTTTTGAGAAC (SEQ ID NO:2).

After PCR with primers of LucP4-EcoRI and LucP2-XhoI, the PCR product (GC-EcoRI site-GCCACC-ATG (Met)-ACT- . . . RLuc . . . -CAA (Gln)-TAA (Stop)-XhoI site-TG was digested with EcoRI and XhoI and cloned into pcDNA3 vector. (See the vector map and sequence, FIG. 1). A Kozak sequence was used for increased (i.e., more effective) expression of luciferase in mammalian cells. The sequence of the vector insert was verified by DNA sequencing from both DNA strands. The cloned luciferase gene matched the reported GenBank sequence.

Transfection of Mammalian Cells

HeLa cells were seeded onto 10 cm plates 24 hours before transfection. Plasmid pcDNA-Rluc or pcDNA3-β-galactosidase was transfected into HeLa cells with Lipofectamine according to manufacture's protocol (GIBCO-BRL). Cells were cultured for an additional 48 hours before activity assay.

HeLa cells were harvested by trypsin/EDTA, washed once with culture medium containing 10% serum and counted using a hemocytometer. The cells were then resuspended at about 1.5×10⁶ cells/ml. The cells were serial-diluted by aliquoting 200 μl the cell suspension into the first row of wells of a 96-well plate and making a series of 4 fold dilutions by transferring 50 μl of cell suspension into the next row of wells which already contained 150 μl of culture medium. The cells were then cultured an additional 24 hours.

To determine the transfection efficiency, about 2×10⁵ cells transfected with pcDNA3-β-galactosidase were seeded onto another 10 cm plate. X-gal staining was performed after 24 hours in culture. Transfected cells (e.g., cells positive for galactosidase) appeared as blue cells.

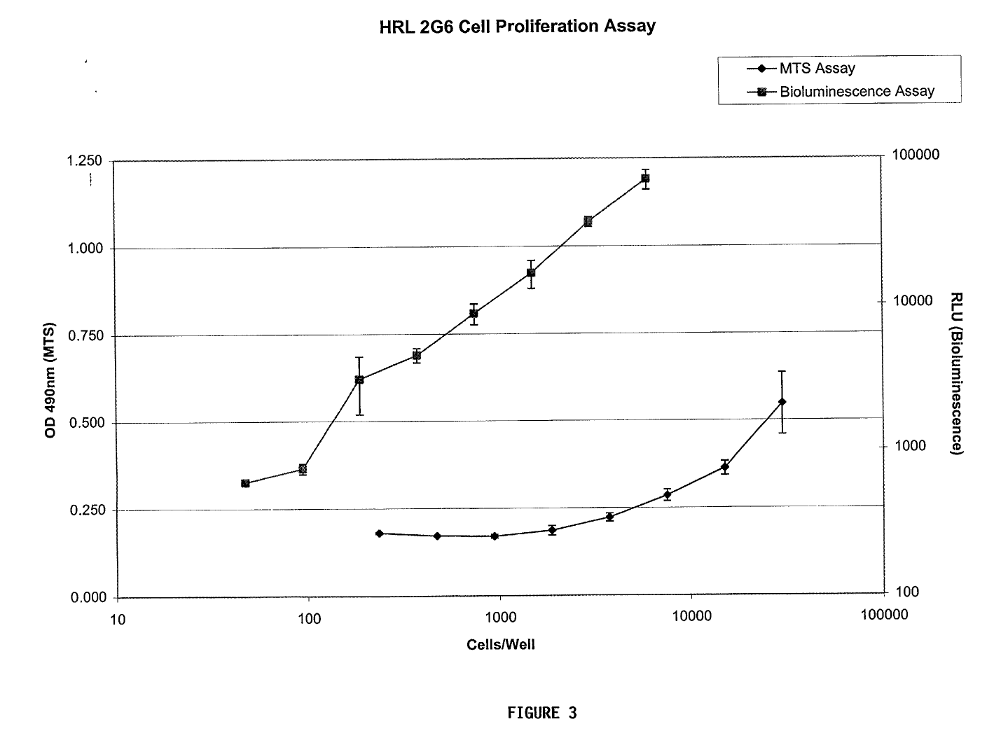

COS-7 cells were transiently transfected with plasmid pcDNA-Rluc or pcDNA3-β-galactosidase using Lipofectamine according to manufacture's protocol (GIBCO-BRL). Similar results to those depicted in FIG. 3 were obtained from these COS-7 cells.

MTT Assays

The MTT Assay was adapted from a method described in Mossmann, Journal of Immunological Methods, 65:55-63, 1983. The MTT assay quantitates the reduction and subsequent trapping of a yellow tetrazolium dye which is reduced by the electron transport chain of functional mitochondria to a purple formazan dye. An MTT assay was carried out using Promega's CELL TITER 96 AQᵤₑₒᵤₛ according to the manufacturer's instuctions. The dye solution was aliquoted into half of the wells of the 96 well plate and incubated at 37° C., 5% CO₂ for 1 hour. Culture medium alone plus the dye was used as a blank. Absorbance of each well was measured at 490 nm.

Assay for Luciferase Activity

Medium from the 96-well plate were aspirated and 20 μl of lysis buffer was added to each well. The 96 well plate was incubated for 20 minutes at room temperature. 5-10 μl of lysate were removed and placed in a clean 96 well plate. 200 μl of luciferase substrate (coelenterazine) at 1 μM was mixed with the lysate and light production was measured for 15 seconds immediately upon the addition of substrate. Wells containing medium only were used as controls.

Cell proliferation assays using Renilla luciferase is 100-300 fold more sensitive than conventional MTT assays. (FIG. 2). The assay was able to detect as little as 50 cells. Experiments using stably transfected HeLa cells demonstrated similar results (FIG. 3).

While the invention has been described in detail with reference to certain preferred embodiments thereof, it will be understood that modifications and variations are within the spirit and scope of that which is described and claimed.

What is claimed is:

1. A method for determining the effect of an agent on cell proliferation, comprising:
contacting a cell containing a Renilla luciferase polypeptide or a polynucleotide encoding a Renilla luciferase with an agent suspected of modulating cell proliferation under conditions that allow the agent and the cell to interact; and
comparing the light emission data from the cell to the light emission data from the cell in the absence of the agent, wherein a difference in light emission data is indicative of an effect on cell proliferation.

2. The method of claim 1, wherein the cell is a prokaryotic cell.

3. The method of claim 1, wherein the cell is a eukaryotic cell.

4. The method of claim 3, wherein the eukaryotic cell is a mammalian cell.

5. The method of claim 4, wherein the mammalian cell is a human cell.

6. The method of claim 1, wherein the cell is a cancer cell.

7. The method of claim 1, wherein the cell contains a transgene encoding Renilla luciferase.

8. The method of claim 7, wherein the cell is a HeLa cell.

9. The method of claim 1, wherein the agent is selected from the group consisting of a peptide, a protein, a chemical, a nucleic acid sequence, a small molecule, and a biological agent.

10. The method of claim 9, wherein the chemical is a drug.

11. The method of claim 10, wherein the drug is an antibiotic.

12. The method of claim 10, wherein the drug is a chemotherapeutic drug.

13. The method of claim 1, wherein the cell is obtained from a subject.

14. The method of claim 13, wherein the subject is a mammal.

15. The method of claim 14, wherein the mammal is a human.

16. The method of claim 1, wherein the modulation is inhibition of cell proliferation.

17. The method of claim 1, wherein the modulation is stimulation of cell proliferation.

18. A method for determining cell proliferation of a cell or population of cells comprising:
obtaining light emission data from a cell containing a Renilla luciferase over a period of time wherein a change in light emission data is indicative of proliferation.

19. The method of claim 18, wherein the cell is a prokaryotic cell.

20. The method of claim 18, wherein the cell is a eukaryotic cell.

21. The method of claim 20, wherein the eukaryotic cell is a mammalian cell.

22. The method of claim 21, wherein the mammalian cell is a human cell.

23. The method of claim 18, wherein the cell is a cancer cell.

24. The method of claim 18, wherein the cell is in a culture of cells.

25. The method of claim 18, wherein the cell contains a transgene encoding Renilla luciferase.

26. The method of claim 25, wherein the cell is a HeLa cell.

27. The method of claim 18, wherein the cell is obtained from a subject.

28. The method of claim 27, wherein the subject is a mammal.

29. The method of claim 28, wherein the mammal is a human.

30. The method of claim 18, wherein the cell is obtained from a tissue sample.

31. A method for determining the effect of an agent on cell proliferation, the method comprising:
transfecting a cell obtained from a sample with a vector containing a polynucleotide sequence encoding a Renilla luciferase;
contacting the transfected cell with an agent suspected of modulating cell proliferation under conditions that allow the agent and the cell to interact; and
comparing the light emission data from the cell to the light emission data from the cell in the absence of the agent, wherein a difference in light emission data is indicative of an effect on cell proliferation.

32. The method of claim 31, wherein the cell is a prokaryotic cell.

33. The method of claim 31, wherein the cell is a eukaryotic cell.

34. The method of claim 33, wherein the eukaryotic cell is a mammalian cell.

35. The method of claim 34, wherein the mammalian cell is a human cell.

36. The method of claim 31, wherein the cell is a cancer cell.

37. The method of claim 31, wherein the sample is obtained from a subject.

38. The method of claim 37, wherein the subject is a mammal.

39. The method of claim 38, wherein the mammal is a human.

40. The method of claim 31, wherein the sample is a biological sample.

41. The method of claim 40, wherein the biological sample is selected from the group consisting of a blood sample, a urine sample, a stool sample, and a tissue sample.

42. The method of claim 31, wherein the agent is selected from the group consisting of a peptide, a protein, a chemical, a nucleic acid sequence, a small molecule and a biological agent.

43. The method of claim 42, wherein the chemical is a drug.

44. The method of claim 43, wherein the drug is an antibiotic.

45. The method of claim 43, wherein the drug is a chemotherapeutic drug.

46. The method of claim 31, wherein the modulating is inhibition of cell proliferation.

47. The method of claim 31, wherein the modulating is stimulation of cell proliferation.

48. A vector containing a polynucleotide sequence encoding a Renilla luciferase for expression in a eukaryotic organism.

49. A eukaryotic host cell containing an expression vector encoding Renilla luciferase.

50. The host cell of claim 49, wherein the host cell is a mammalian cell.

51. The host cell of claim 50, wherein the mammalian cell is a human cell.

52. The host cell of claim 51, wherein the human cell is a HeLa cell.

53. The host cell of claim 52, wherein the HeLa cell has ATCC accession number X.

54. The host cell of claim 49, wherein the cell is stably transfected with the Renilla luciferase.

55. The host cell of claim 49, wherein the cell is transiently transfected with the Renilla luciferase.

56. A method of diagnosing a cell proliferative disorder, comprising:
transfecting a cell obtained from a subject with a vector containing a polynucleotide encoding a Renilla luciferase;
obtaining light emission data from the cell over a period of time; and comparing the light emission data from the cell to light emission data from a cell which does not have a cell proliferative disorder, wherein a difference in light emission is indicative of a cell proliferative disorder.

57. The method of claim 56, wherein the cell proliferative disorder is a neoplasm or a cancer.

58. The method of claim 56, wherein the cell is obtained from a tissue.

59. The method of claim 56, wherein the cell is a mammalian cell.

60. The method of claim 59, wherein the mammalian cell is a human cell.

61. The method of claim 56, wherein the light emission data is obtained continuously over a period of time.

62. The method of claim 56, wherein the light emission data is obtained at two or more time points.

63. A method of screening mammalian cells to determine their susceptibility to treatment with an agent, comprising:
contacting cells containing a Renilla luciferase with an agent; and
measuring light emissions from the cells in the presence and absence of the agent, wherein a difference in light emissions is indicative of an agent which affects cell proliferation.

64. The method of claim 63, wherein the cells are obtained from a subject.

65. The method of claim 64, wherein the subject is a human.

66. The method of claim 63, wherein the agent is selected from the group consisting of a peptide, a protein, a chemical, a nucleic acid sequence, a small molecule, and a biological agent.

67. The method of claim 63, wherein the agent is a drug.

68. The method of claim 67, wherein the agent is an antibiotic or a chemotherapeutic agent.

69. A kit comprising a container containing a host cell of claim 49 and instructions for use of the cell for measuring cell proliferation.

70. The kit of claim 69, further comprising a container containing coelenterazine.
